(19) United States
(12) Patent Application Publication
(10) Pub. No.: US 2021/0077369 A1
(43) Pub. Date: Mar. 18, 2021

(54) PHOTOSTABLE COMPOSITION MADE FROM PERLITE/TITANIUM/SILICA COMPOSITE PARTICLES

(71) Applicant: L'OREAL, Paris (FR)

(72) Inventors: Didier CANDAU, Chevilly Larue (FR); Anne MAGNAN, Chevilly Larue (FR); Mahassine SAFOUANE, Chevilly Larue (FR)

(21) Appl. No.: 16/612,558

(22) PCT Filed: May 11, 2018
(86) PCT No.: PCT/EP2018/062204

§ 371 Date: Dec. 6, 2019

(30) Foreign Application Priority Data
May 12, 2017 (FR) 1754213

(51) Int. Cl. A61K 8/29; A61Q 17/04; A61K 8/11; A61K 8/25; A61K 8/96; A61K 8/27; A61K 8/19

(52) U.S. Cl. CPC A61K 8/29; A61Q 17/04; A61K 8/11; A61K 8/19; A61K 8/965; A61K 8/27; A61K 8/25

(57) ABSTRACT

This invention relates to a composition comprising, particularly in a cosmetically acceptable medium:
a) at least one aqueous phase; and
b) particles with a number-average elementary size greater than 0.1 μm comprising:
i) a perlite matrix,
ii) at least one inorganic UV filter, and
iii) a silica coating, said silica representing at least 1% by weight relative to the total weight of the composite particle.

It also relates to a makeup or treatment process using such a composition.

This invention relates to a composition comprising, particularly in a cosmetically acceptable medium:

a) at least one aqueous phase; and
b) particles with a number-average elementary size greater than 0.1 μm comprising:
i) a perlite matrix,
ii) at least one inorganic UV filter, and
iii) a silica coating, said silica representing at least 1% by weight relative to the total weight of the composite particle.

It is known that light rays of wavelength between 280 nm and 400 nm enable tanning of the human epidermis. However, rays having wavelengths more particularly between 280 and 320 nm, known as UV-B, cause skin erythema and burns which can impede natural tan development.

For these reasons and for esthetic reasons, there is a constant demand for means for controlling natural tanning with a view to controlling skin color: it is thus necessary to filter UV-B radiation.

It is also known that UV-A rays, having wavelengths between 320 and 400 nm, which cause the skin to tan, are liable to induce damage thereof, particularly in the case of sensitive skin or skin continually exposed to solar radiation. UV-A rays particular cause a loss of skin elasticity and the appearance of wrinkles giving rise to premature skin ageing.

Thus, it is also desirable to filter UV-A radiation.

Numerous photoprotective compositions have been proposed to date to protect the effects induced by UVA and/or UVB. These compositions generally contain organic or mineral filters, more particularly mixtures of organic liposoluble filters and/or water-soluble filters, combined with metal oxide pigments such as titanium dioxide or zinc oxide. These inorganic particles make it possible to increase sun protection, which reduces the quantity of organic filters and thus enhances the cosmeticity of the formulas.

While mineral filters such as titanium dioxide or zinc oxide are widely used in cosmetics for the UV-absorbing properties thereof, they however cause whitening when applied to the skin, which is not esthetic.

Furthermore, absorption in UV induces undesirable oxidation-reduction phenomena of these mineral filters, which are manifested by a change of color of the composition. Indeed, blue or yellow discoloration and oxidation of the other constituents are observed. This is referred to as photocatalytic activity. This photocatalytic activity may cause undesirable reactions for the skin and for the constituents present in the formulation.

In order to reduce the photocatalytic activity while retaining effective UV absorption, an organic or mineral coating may be used for these mineral filters. Among the coatings routinely used, silica or alumina are essentially found, but also organic substances such as cyclomethicone, dimethicone or stearic acid. This is for example described in U.S. Pat. No. 5,562,990, which discloses fluorinated titanium dioxide particles comprising a layer of alumina, said particles being treated with an organosilicon compound. These treatments have an undesirable environmental profile, in particular the treatments with cyclomethicone, dimethicone or organosilicons.

There is a constant need for effective photoprotective compositions that are photostable, and which exhibit reduced whitening on application.

The inventors have discovered that using composite particles made from TiO2 and perlite and protected by a surface treatment consisting of silica, it was possible to obtain photoprotective compositions particularly stable in UV rays and not causing oxidation of the other constituents present in the composition. Such compositions exhibit effective sun protection, and are less white (reduced whitening) on application.

This invention thus relates to a composition comprising, particularly in a cosmetically acceptable medium:

a) at least one aqueous phase; and
b) particles with a number-average elementary size greater than 0.1 μm comprising:
i) a perlite matrix,
ii) at least one inorganic UV filter, and
iii) a silica coating, said silica representing at least 1% by weight relative to the total weight of the composite particle.

It also relates to a cosmetic process for the treatment and/or makeup of human keratin materials, particularly the skin of the body or face or hair, comprising at least the application on said keratin materials of a composition as defined above.

The term “human keratin materials” refers to the skin (body, face, eye contour), hair, eyelashes, eyebrows, body hair, nails, lips and/or mucous membranes.

The term “cosmetically acceptable medium” refers to any medium compatible with the skin and/or integuments thereof, having a pleasant color, odor and texture and not giving rise to unacceptable discomfort (tingling, tightness, redness), liable to dissuade the consumer from using the composition.

The term “inorganic UV filter” refers to a molecule not comprising carbon atoms in the structure thereof and capable of filtering UV radiation between 280 and 400 nm.

The term “average size” refers to the number-average size or the average diameter in number of particles. The average particle sizes can be measured by static light scattering by means of a Malvern MasterSizer 2000 laser granulometer. Data are processed based on Mie's scattering theory. This theory, precise for isotropic particles, can determine an “effective” particle diameter in the case of non-spherical particles. This theory is described particularly in the book by Van de Hulst, H. C., “Light Scattering by Small Particles”, Chapters 9 and 10, Wiley, New York, 1957.

The term “average elementary size” refers to the non-aggregated particle size.

Composite Particles

The composite particles according to the invention comprise a matrix consisting of particles of perlite and at least one inorganic UV filter.

The composite particles suitable for use according to the invention may be monolayer or multilayer.

The composite particles suitable for use according to the invention may be spherical or non-spherical, preferably non-spherical.

The term “non-spherical” refers to three-dimensional particles (length, width, thickness or height) for which the ratio of the greatest dimension to the smallest dimension is greater than 1.2. They comprise particles of parallelepipedal (rectangular or square surface), discoidal (circular surface) or ellipsoidal (oval surface) shape, characterized by three dimensions: a length, a width and a height. When the shape is circular, the length and the width are identical and correspond to the diameter of a disk, whereas the height corresponds to the thickness of the disk. When the surface is oval, the length and the width correspond respectively to the major axis and to the minor axis of an ellipse and the height corresponds to the thickness of the elliptical disk formed by the sheet. When it consists of a parallelepiped, the length and the width may be of identical or different sizes: when they are of the same size, the shape of the surface of the parallelepiped is square; otherwise, the shape is rectangular. As regards the height, it corresponds to the thickness of the parallelepiped.

According to a first alternative embodiment, the composite particles contain a matrix consisting of perlite particles and wherein inorganic UV filter particles are included. According to this embodiment, the matrix has inclusions and inorganic UV filter particles are placed in the matrix inclusions.

Preferentially, the composite particles according to the invention have a number-average elementary size varying between 0.1 and 30 μm, preferably between 1 and 28 μm, more preferentially between 3 and 25 μm.

Preferentially, when the composite particles according to the invention are spherical, the number-average elementary size thereof varies between 0.1 and 30 μm.

Preferentially, when the composite particles according to the invention are non-spherical, they are characterized by a number-average elementary size of 0.1 to 30 μm.

The non-spherical composite particles suitable for use according to the invention will preferably be in sheet form. The term “sheet form” refers to a parallelepipedal form.

The composition particles in sheet form preferably have a number-average elementary size of 0.1 to 30 μm.

The composite particles according to the invention are preferably present, in the compositions according to the invention, in concentrations ranging from 1 to 70%, preferably from 1.5 to 50%, and preferentially from 2 to 40% by weight relative to the total weight of the composition.

Inorganic UV Filters

The composite particles according to the invention comprise at least one inorganic UV filter.

The inorganic UV filters are preferentially metal oxide pigments. More preferentially, the inorganic UV filters according to the invention are metal oxide particles having an average elementary particle size less than 0.1 μm.

The inorganic UV filter is generally chosen among metal oxides, preferably titanium, zinc, iron oxides or mixtures thereof, and more particularly among titanium dioxide, zinc oxide and mixtures thereof. Particularly preferably, the inorganic UV filter is titanium dioxide (TiO2).

In particular, the titanium dioxide (TiO2) may be in rutile and/or anatase form and/or in an amorphous form.

The mass content of inorganic UV filter in the composite particles according to the invention is preferably from 1% to 70% by weight, more preferably from 1.2% to 65% by weight, and even more preferably from 1.5% to 60% by weight relative to the total weight of a composite particle.

Perlite Particles

The composite particles according to the invention comprise a matrix consisting of particles of perlite.

Perlite is a natural glass of volcanic origin, glossy light gray or black in color, resulting from the rapid cooling of lava and presented in the form of small particles resembling pearl.

The perlites suitable for use according to the invention are generally aluminosilicates of volcanic origin and have by way of composition:

70.0-75.0% by weight of silica SiO2

12.0-15.0% by weight of aluminum oxide Al2O3

3.0-5.0% of sodium oxide Na2O

3.0-5.0% of potassium oxide K2O

0.5-2% of iron oxide Fe2O3

0.2-0.7% of magnesium oxide MgO

0.5-1.5% of calcium oxide CaO, and

0.05-0.15% of titanium oxide TiO2.

Preferentially, the perlite particles used according to the invention will be in porous expanded form.

The perlite is milled, dried then calibrated in a first step. The product obtained known as Perlite Ore is gray in color and of the order of 100 μm in size. The Perlite Ore is then expanded (1000° C./2 seconds) to produce more or less white particles. When the temperature attains 850-900° C., the water trapped in the structure of the material evaporates and induces the expansion of the material relative to the original volume thereof. The expanded perlite particles according to the invention may be obtained by means of the expansion process described in U.S. Pat. No. 5,002,698.

Preferably, the particles of perlite used will be milled; they are in this case referred to as Expanded Milled Perlite (EMP).

They preferably have a particle size defined by a median diameter D50 ranging from 0.5 to 50 μm and preferably from 0.5 to 40 μm.

Preferably, the perlite particles according to the invention have a particle size distribution such that at least 50% of the particles have a size less than 20 μm. Furthermore, they preferentially have a particle size distribution such that 90% by weight of the particles have a size less than 55 μm and preferably a size less than 40 μm. It is preferred moreover than 90% by weight of the particles have a size greater than 5 μm.

Preferably, the particles of perlite used have a loose bulk apparent density at 25° C. ranging from 10 and 400 kg/m3 (Standard DIN 53468), and preferably from 10 and 300 kg/m3.

Expanded perlite particles sold under the trade names OPTIMAT 1430 OR® or OPTIMAT 2550® by the company WORLD MINERALS or the commercial products GK-110 THIN® and GK-110 EXTRA THIN® by the company LANGFANG XINDAZHONG FILTER and the company HENAN ZHONGNAN FILTER AID will be used in particular.

The mass content of perlite in the composite particles according to the invention is preferably from 10 to 99% by weight, more preferentially from 20 to 98% by weight, and even more preferentially from 30 to 97% by weight relative to the total weight of a composite particle.

Composite Particle Preparation

The composite particles according to the present invention may be obtained according to the process described in patent FR2882371B1 consisting of impregnating particles of perlite with an aqueous suspension of particles of metal oxide such as titanium dioxide having a number-average elementary particle size less than 0.1 μm, then reducing the precursors within said material forming the matrix; the impregnation being carried out under saturating vapor pressure and under reflux of the solution of one or a plurality of precursors of metal oxide (for example titanium dioxide) and the reduction being carried out by a radiolytic process.

The metal oxide precursors may be chosen among mineral salts (for example sulfates, perchlorates), organic salts (for example formiates, neodecanoates) or organometallic compounds.

The solution of precursor(s) may further contain an oxidizing radical intercepting agent, which intercepts the oxidizing radicals formed in the solution during irradiation, which prevents oxidation of the colloidal particles produced. The oxidizing radical interceptor is chosen preferably among primary alcohols, secondary alcohols and formiates.

The composite particles made from perlite and inorganic filter may also be obtained according to the process described in application WO 2006/083326 which consists of generating a gaseous dispersion in a flow comprising droplets of a precursor substrate dispersed in gaseous phase. The precursor substrate contains a liquid medium and at least one metal oxide precursor of elementary size less than 0.1 μm and a matrix made from perlite. The composite particles are formed from the gaseous dispersion by removing a portion of the liquid vehicle of the droplets of the precursor medium.

According to a particularly preferred embodiment of the invention, the composite particles may also be prepared according to the process described in application KR1020000069638 which consists, according to a sol-gel method, of mixing in water an aqueous suspension of metal alkoxide (particularly titanium alkoxide such as titanium tetra-n-butyl oxide also known as tetra-n-butyl-titanate (TNBT)) with a perlite matrix and reacting by hydrolysis the metal alkoxide for a time ranging preferably from 1 to 8 hours at a temperature ranging preferably from 30 to 78° C. Then, the suspension obtained is filtered, washed and dried to produce a composite power of perlite coated with metal oxide. To enhance the uniformity of the coating of inorganic filter on the perlite particles, the composite particles obtained with the metal alkoxide suspension may be reacted according to the same process.

According to a further particular embodiment of the invention, the composite particles may also be prepared according to the process described in U.S. Pat. No. 6,447,759, which consists of preparing a suspension of perlite particles, by adding a complexing agent to the suspension, by adding a precursor metal oxide salt of the inorganic filter and an alkaline agent of the carbonate type to form basic metal oxide carbonate particles on the perlite matrix and calcining the composite particles.

According to a particular embodiment of the invention, the composite particles may also be prepared according to the process described in application JP2008115161, consisting of preparing a suspension of perlite particles at acidic pH in the presence of hydrochloric acid. The metal oxide particles forming the inorganic filter are then added. At acidic pH, a flocculent such as calcium chloride is added to obtain the composite particles.

According to a further particular embodiment of the invention, the composite particles according to the invention may be prepared by subjecting the particles of perlite and the inorganic filter(s) as defined above to a mechanochemical fusion process.

A mechanochemical fusion process refers to a process wherein a mechanical force such as a shock, a friction force, a shear force is applied to a plurality of compounds, to produce partial fusion of the various compounds.

The mechanochemical fusion process may be implemented, for example, with an apparatus comprising a rotary chamber and an internal fixed part with a scraper, such as the mechanofusion device under the trade name 20 Hosokawa Micron Corporation® in Japan.

It is preferable to use a so-called hybridizer process as the mechanochemical fusion process.

The hybridizer process was developed in the 1980s. The hybridizer process is a category of mechanochemical fusion processes wherein a high mechanical force is applied to a plurality of particles to produce a mechanochemical reaction and form a composite particle.

According to the hybridizer process, the mechanical force is applied by a high-speed rotor suitable for having a diameter of 10 cm to 1 m, and may rotate at a speed of 1000 rpm to 100,000 rpm. Thus, the hybridizer process may be defined as a mechanochemical fusion process using a high-speed rotor. The hybridizer process is carried out in the presence of air or under dry conditions. Indeed, due to the high speed of the rotor, a high-speed air flow may be produced near the rotor. Some liquid materials may be subjected to the hybridizer process with solid materials. The term “hybridizer process” has been used as a technical term in the present description.

The hybridizer process may be carried out using a hybridization system of the Nara Machinery® make in Japan, wherein the particles of perlite and those of inorganic filter are introduced into a hybridizer equipped with a high-speed rotor provided with a plurality of blades in a chamber under dry conditions. The particles are dispersed in the chamber and a mechanical and thermal energy (i.e. compression, friction, shear stress) are applied to the particles for a short period of time such as from 1 to 10 minutes, preferably from 1 to 5 minutes. One type of particles (i.e. fine particles) incorporated or fixed on another type of particles (core particles) to form the composite particles according to the invention is obtained. It is preferable that the particles be subjected to one or a plurality of electrostatic treatments such as oscillation to form an “orderly mixture” wherein one type of particles is spread to coat the other type of particles.

The hybridizer process may also be carried out using a theta composter of the Tokuju Corporation@ make in Japan. The hybridizer process may be carried out with Composi Hybrid or Mechano Hybrid device sold by the company Nippon Coke.

According to the present invention, the particles of perlite, the inorganic filter(s) may be introduced into a hybridizer to form a composite pigment. The hybridizer process may be carried out using a rotor rotating at 8000 rpm (100 m/sec) for about 3 minutes.

Furthermore, the hybridizer process can create an orderly assembly (uniform coating) of the inorganic UV filter(s) on the particle of perlite and produce strong bonds on the surface of the particle of perlite and the layer of coating comprising the inorganic UV filter(s).

It should be noted that the hybridizer process is very different from other processes using for example a bead mill and a fluid-energy mill. Indeed, bead mills cause pulverization or agglomeration of the particles forming the core of the composite and fluid-energy mills cause pulverization of said particles rendering uniform coating of the fine particles on the particles forming the core of the composite difficult.

Silica Coating

The composite particles according to the invention also comprise a silica coating, which represents at least 1% by weight relative to the total weight of the composite particle. This silica forms the coating of the composite particles. It is therefore distinct from the silica comprised in the perlite matrix.

Indeed, the composite particles according to the invention have typically undergone one or a plurality of surface treatments of chemical, electronic, mechanochemical and/or mechanical nature with compounds made from silica (SiO2).

Thus, the composite particles according to the invention typically comprise a perlite matrix, at least one inorganic UV filter, and have undergone a surface treatment (or coating) with silica, this silica representing at least 1% by weight relative to the total weight of the composite particle.

The mass content of silica (i.e. the coating) in the composite particles according to the invention is preferably at least 2% by weight, preferably between 1 and 10% by weight, more preferentially between 2 and 8% by weight, and even more preferentially between 2.5 and 6% by weight relative to the total weight of a composite particle.

According to an embodiment, once the composite particles comprising the perlite matrix and the inorganic UV filter are obtained, particularly by means of one of the processes described above, they undergo a surface treatment with silica, this silica representing at least 1% by weight relative to the total weight of the composite particle. The particles treated in this way are then washed, dried and filtered.

Preferably, the composite particles according to the invention may be prepared according to one of examples A or B:

### Preparation Example A

100 g of perlite powder sold under the trade name GK-110 Thin® by the company Langfang Xindazhong Filter and the company Henan Zhongnan Filter Aid was placed in a round-bottomed flask equipped with a stirrer and 150 g of titanium tetra-n-butoxide (Super Urecoat Industries) in suspension form was added. The perlite was designed to absorb the titanium precursor. A quantity of 0.2 to 1 liter of water was then added to enable a reaction of 1 to 8 hours at 30-100° C. Then, the suspension obtained was filtered, washed and dried to produce a composite perlite powder coated with titanium dioxide.

In a second step, 100 g of this composite powder was placed in a round-bottomed flask and 150 g of titanium tetra-n-butoxide in suspension form was added. The composite was then embodied to absorb the titanium precursor. A quantity of 0.2 to 1 liter of water was then added to enable a reaction of 1 to 8 hours at 30-100° C. Then, the suspension obtained was filtered, washed and dried to produce a composite perlite powder coated with titanium dioxide. Finally, a composite powder containing 40% by weight of titanium dioxide was obtained.

This composite powder was then coated with at least 1% silica by weight relative to the total weight of the composite particle.

### Preparation Example B

A composite powder made from perlite and titanium dioxide was prepared, under the same conditions as in example A, using a perlite powder sold under the trade name GK-110 Extra Thin by the company Langfang Xindazhong Filter and the company Henan Zhongnan Filter Aid.

This composite powder was then coated with at least 1% silica by weight relative to the total weight of the composite particle.

Aqueous Phase

The compositions according to the invention comprise at least one aqueous phase.

The aqueous phase contains water and possibly at least one organic solvent that is or miscible in water.

An aqueous phase suitable for the invention may for example comprise water chosen from among a natural spring water, for example such as La Roche-Posay water, Vittel water, or Vichy water, or a floral water.

The water-soluble or water-miscible solvents suitable for the invention comprise short-chain mono-alcohols for example C1-C4 such as ethanol, isopropanol; diols or polyols such as ethylene glycol, 1,2-propylene glycol, 1,3-butylene glycol, hexylene glycol, diethylene glycol, dipropylene glycol, 2-ethoxyethanol, diethylene glycol monomethylether, triethylene glycol monomethylether, glycerol, sorbitol and mixtures of them.

According to one preferred embodiment, in particular ethanol, propylene glycol, glycerin or one of their mixtures can be used.

According to a particular embodiment of the invention, the aqueous phase represents from 15 to 95% by weight, preferentially from 30 to 80% by weight, more preferably from 40 to 70% by weight relative to the total weight of the composition.

Oily Phase

The compositions according to the invention comprise at least one oily phase.

For the purposes of this invention, “oily phase” denotes a phase comprising at least one oil and all liposoluble and lipophilic ingredients and fatty substances used for the formulation of compositions according to the invention.

Oil denotes any fatty substance in liquid form at ambient temperature (20-25° C.) and at atmospheric pressure (760 mm Hg). An oil according to the invention may be volatile or non-volatile.

The oil may be chosen from hydrocarbon oils, silicon oils, fluorinated oils and mixtures thereof. A hydrocarbon oil suitable for the invention may be an animal hydrocarbon oil, a plant hydrocarbon oil, a mineral hydrocarbon oil, or a synthetic hydrocarbon oil. An oil suitable for the invention may advantageously be chosen from mineral hydrocarbon oils, plant hydrocarbon oils, synthetic hydrocarbon oils, silicon oils and mixtures thereof.

For the purposes of this invention, the term “silicone oil” denotes an oil comprising at least one silicon atom, and in particular at least one SiO group.

The term “hydrocarbon oil” is intended to denote an oil containing mainly hydrogen and carbon atoms.

The term “fluorinated oil” denotes an oil comprising at least one fluorine atom.

A hydrocarbon oil according to the invention may also possibly comprise oxygen, nitrogen, sulfur and/or phosphorus atoms, for example in the form of hydroxyl, amine, amide, ester, ether or acid groups, and in particular in the form of hydroxyl, ester, ether or acid groups.

The oily phase generally comprises, besides the lipophilic UV filter(s), at least one volatile or non-volatile hydrocarbon oil and/or a volatile and/or non-volatile silicone oil.

The term “volatile oil” according to the invention denotes any oil capable of evaporating in contact with skin or keratin fiber, in less than one hour, at ambient temperature and atmospheric pressure. The volatile oil(s) according to the invention is (are) volatile cosmetic oils that are liquid at ambient temperature, having a vapor pressure different from zero at ambient temperature and atmospheric pressure, particularly ranging from 0.13 Pa to 40,000 Pa (10⁻³ to 300 mm Hg), particularly ranging from 1.3 Pa to 13,000 Pa (0.01 to 100 mm Hg), and more specifically ranging from 1.3 Pa to 1300 Pa (0.01 to 10 mm Hg).

The term “non-volatile oil” denotes an oil remaining on skin or keratin fiber at ambient temperature and atmospheric pressure for at least several hours and particularly having a vapor pressure less than 10⁻³ mm Hg (0.13 Pa).

Hydrocarbon Oils

Among non-volatile, non-hydrocarbon oils that can be used according to the invention, mention may be made in particular of:

(i) hydrocarbon oils of plant origin such as triglyceride esters which are generally triesters of fatty acids and glycerol for which the fatty acids can have chain lengths ranging from C4 to C24, with the latter able to be linear or branched, saturated or unsaturated; these oils are in particular wheat germ, sunflower, grape seed, sesame, corn, apricot, castor, shea, avocado, olive, soybean oils, sweet almond, palm, rapeseed, cotton, hazelnut, macadamia, jojoba, alfalfa, poppy seed, pumpkin, sesame, squash, rapeseed, blackcurrant, evening primrose, millet, barley, quinoa, rye, safflower, candlenut, passiflora, musk rose oil; or caprylic/capric acid triglycerides such as those sold by Stearineries Dubois or those sold under the trade names Miglyol 810, 812 and 818 by the company Dynamit Nobel;

(ii) synthetic ethers having from 10 to 40 carbon atoms;

(iii) linear or branched hydrocarbons of mineral or synthetic origin, such as petroleum jelly, polydecenes, hydrogenated polyisobutene such as Parleam, squalane and mixtures thereof;

(iv) synthetic esters such as oils having the formula RCOOR′ in which R represents the residue of a linear or branched fatty acid comprising 1 to 40 carbon atoms and R′ represents a hydrocarbon chain, particularly branched containing 1 to 40 carbon atoms on the condition that R+R′ is >10, such as for example Purcellin oil (cetostearyl octanoate), isopropyl myristate, isopropyl palmitate, C12 to C15 alcohol benzoates like the product sold under the trade name “Finsolv TN” or “Witconol TN” by WITCO or “TEGOSOFT TN” by EVONIK GOLDSCHMIDT, 2-ethylphenyl benzoate such as the commercial product sold under the name “X-TEND 226” by the company ISP, isopropyl lanolate, hexyl laurate, diisopropyl adipate, isononyl isononanoate, oleyl erucate, 2-ethylhexyl palmitate, isostearyl isostearate, diisopropyl sebacate like the product sold under the trade name “Dub Dis” by the company Stearinerie Dubois, octanoates, decanoates or ricinoleates of alcohols or polyalcohols such as propylene glycol dioctanoate; hydroxylated esters, such as isostearyl lactate, diisostearyl malate; pentaerythritol esters; citrates or tartrates such as C12-C13 linear di-alkyl tartrates like those sold under the name COSMACOL ETI by ENICHEM AUGUSTA INDUSTRIALE and C14-C15 linear di-alkyl tartrates like those sold under the name COSMACOL ETL by the same company; acetates;

(v) fatty alcohols that are liquid at ambient temperature, with a branched and/or unsaturated carbon chain having 12 to 26 carbon atoms, such as octyldodecanol, isostearyl alcohol, oleic alcohol, 2-hexyldecanol, 2-butyloctanol, 2-undecylpentadecanol;

(vi) higher fatty acids, such as oleic acid, linoleic acid, linolenic acid;

(vii) carbonates such as dicaprylyl carbonate like the product sold under the name “Cetiol CC” by the company Cognis;

(viii) fatty amides such as isopropyl N-lauroyl sarcosinate like the product sold under the trade name Eldew SL-205 by Ajinomoto and mixtures thereof.

Among the non-volatile hydrocarbon oils that can be used according to the invention, triglyceride esters and particularly caprylic/capric acid triglycerides, synthetic and particularly isononyl isononanoate, diisopropyl sebacate, benzoate of C12-C15 alcohols, 2-ethylphenyl benzoate and fatty acids particularly octyldodecanol will be more particularly preferred.

Volatile hydrocarbon oils that can be used according to the invention can be chosen particularly from among hydrocarbon oils having 8 to 16 carbon atoms, and particularly branched C8-C16 alkanes such as petroleum-based C8-C16 isoalkanes (also referred to as isoparaffins) such as isododecane (also referred to as 2,2,4,4,6-pentamethylheptane), isodecane, isohexadecane, oils sold under the trade names Isopars or Permetyls, branched C8-C16 esters, iso-hexyl neopentanoate, and mixtures thereof.

Mention can also be made of the alkanes described in patent applications WO 2007/068371, or WO2008/155059 made by the company Cognis (separate alkane mixtures and different by at least one carbon). These alkanes are obtained from fatty alcohols, themselves obtained from coconut or palm oil. Mention can be made of mixtures of n-undecane (C11) and n-tridecane (C13) obtained in examples 1 and 2 in application WO2008/155059 made by the company Cognis.

Mention can also be made of n-dodecane (C12) and n-tetradecane (C14) sold by Sasol under references PARAFOL 12-97 and PARAFOL 14-97 respectively, and mixtures thereof.

Further volatile hydrocarbon oils such as petroleum distillates, particularly those sold under the name Shell Solt by the company SHELL, may also be used.

In one embodiment, the volatile solvent is chosen from among volatile hydrocarbon oils having 8 to 16 carbon atoms and mixtures thereof.

Silicone Oils

The non-volatile silicone oils can be chosen particularly from among non-volatile polydimethylsiloxanes (PDMS), polydimethylsiloxanes comprising alkyl or alkoxy groups that are pendant and/or at the end of the silicone chain, groups each having 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyl-trisiloxanes or (2-phenylethyl)trimethylsiloxysilicates.

Volatile silicone oils that can be used include for example volatile linear or cyclic silicone oils, particularly those having a viscosity of 8 centistokes (8×10⁻⁶ m²/s), and in particular having 2 to 7 silicon atoms, these silicones optionally comprising alkyl or alkoxy groups having 1 to 10 carbon atoms. Mention may be made, as a volatile silicone oil suitable for use in the invention, in particular, of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

Mention may also be made of volatile alkyl trisiloxane linear oils with general formula (I):

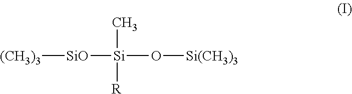

where R represents an alkyl group comprising 2 to 4 carbon atoms and in which one or several hydrogen atoms can be substituted by a fluorine or chlorine atom.

Among oils with general formula (I), mention may be made of: 3-butyl 1,1,1,3,5,5,5-heptamethyl trisiloxane, 3-propyl 1,1,1,3,5,5,5-heptamethyl trisiloxane, and 3-ethyl 1,1,1,3,5,5,5-heptamethyl trisiloxane, corresponding to oils with formula (I) for which R is a butyl group, a propyl group or an ethyl group respectively.

Fluorinated Oils

It would also be possible to use volatile fluorinated oils such as nonafluoromethoxybutane, nonafluoromethoxybutane, decafluoropentane, tetradecafluorohexane, dodecafluoropentane and mixtures thereof.

The oily phase may also comprise other fatty substances. Another fatty substance that can be present in the oily phase can be, for example:
a fatty acid chosen among fatty acids comprising 8 to 30 carbon atoms, such as stearic acid, lauric acid, palmitic acid and oleic acid;
a wax chosen among waxes such as lanolin, beeswax, Carnauba or Candellila wax, paraffin or lignite waxes or microcrystalline waxes, ceresin or ozokerite, synthetic waxes such as polyethylene waxes, Fischer-Tropsch waxes;
a gum chosen among silicone gums (dimethiconol),
a pasty compound, such as optionally polymeric silicone compounds, oligomeric glycerol esters, arachidyl propionate, fatty acid triglycerides and derivatives thereof,
and mixtures thereof.

Preferably, the oily phase represents from 5 to 95% by weight, and preferentially from 10 to 80% by weight relative to the total weight of the composition.

The compositions according to the invention may also further comprise at least one organic UV filter.

Organic UV Filters

The organic UV filters are particularly chosen among cinnamic compounds; anthranilate compounds; salicylic compounds, dibenzoylmethane compounds, benzylidene camphor compounds; benzophenone compounds; diphenylacrylate compounds; triazine compounds; benzotriazole compounds; benzalmalonate compounds particularly those cited in the U.S. Pat. No. 5,624,663; benzimidazole derivatives; imidazoline compounds; bis-benzoazolyl compounds as described in patents EP669323 and U.S. Pat. No. 2,463,264; p-aminobenzoic compounds (PABA); methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; filter polymers and filter silicones such as those described particularly in application WO93/04665; a-alkylstyrene-derived dimers as described particularly in patent application DE19855649; 4,4-diarylbutadiene compounds as described in applications EP0967200, DE19746654, DE19755649, EP-A1008586, EP1133980 and EP133981 and mixtures thereof.

Examples of organic photoprotective agents include those referred to hereinafter using their INCI name:

Cinnamic Compounds:

Ethylhexyl Methoxycinnamate particularly sold under the trade name PARSOL MCX by DSM Nutritional Products

Isopropyl Methoxycinnamate

Isoamyl p-Methoxycinnamate sold under the trade name NEO HELIOPAN E 1000 by Symrise

DEA Methoxycinnamate

Diisopropyl Methylcinnamate

Glyceryl Ethylhexanoate Dimethoxycinnamate.

Para-Aminobenzoic Compounds:

PABA,

Ethyl PABA,

Ethyl Dihydroxypropyl PABA,

Ethylhexyl Dimethyl PABA particularly sold under the name “ESCALOL 507®” by ISP,

Glyceryl PABA,

PEG-25 PABA sold under the name “UVINUL P25®” by BASF.

Salicylic Compounds:

Homosalate sold under the name “Eusolex HMS®” by Rona/EM Industries,

Ethylhexyl Salicylate sold under the name “NEO HELIOPAN OS®” by Symrise,

Dipropyleneglycol Salicylate sold under the name “DIPSAL®” by SCHER,

TEA Salicylate, sold under the name “NEO HELIOPAN ISO” by Symrise.

Dibenzoylmethane Compounds

Butylmethoxydibenzoylmethane or Avobenzone particularly sold under the trade name PARSOL 1789 by DSM Nutritional Products

β,β-Diphenylacrylate Products:

Octocrylene particularly sold under the trade name “UVINUL N 539” by 40 BASF,

Etocrylene, particularly sold under the trade name “UVINUL N 35” by BASF.

Benzophenone Compounds:

Benzophenone-1 sold under the trade name “UVINUL 400” by BASF,

Benzophenone-2 sold under the trade name “UVINUL D 50” by BASF,

Benzophenone-3 or Oxybenzone, sold under the trade name “UVINUL M 5 40” by BASF,

Benzophenone-4 sold under the trade name “UVINUL MS 40” by BASF,

Benzophenone-5 Benzophenone-6 sold under the trade name “Helisorb 11®” by Norquay

Benzophenone-8 sold under the trade name “Spectra-Sorb UV-24®” by 10 American Cyanamid;

Benzophenone-9 sold under the trade name “UVINUL DS 49®” by BASF,

Benzophenone-12 n-hexyl 2-(4-diethylamino-2-hydroxybenzoyl)-benzoate sold under the trade name “UVINUL A Plus C” or mixed with octylmethoxycinnamate 15 sold under the trade name “UVINUL A Plus B®” by the company BASF,

1,1′-(1,4-piperazinediyl)bis[14244-(diethylamino)-2-hydroxybenzoyl]phenyl]-methanone (CAS 919803-06-8) as described in application WO2007/071584; this compound advantageously being used in micronized form (average size from 0.02 to 2 μm) that can be obtained for example using the micronization process described in applications GB-A-2 303 549 and EP-A-893119 and particularly in the form of an aqueous dispersion.

Benzylidene Camphor Compounds:

3-Benzylidene camphor manufactured under the name “MEXORYL SD” by CHIMEX,

4-Methylbenzylidene camphor sold under the name “EUSOLEX 6300®” by MERCK,

Benzylidene Camphor Sulfonic Acid manufactured under the name “MEXORYL SLC” by CHIMEX,

Camphor Benzalkonium Methosulfate manufactured under the name “MEXORYL SO®” by CHIMEX,

Terephthalylidene Dicamphor Sulfonic Acid manufactured under the name “MEXORYL SX®” by CHIMEX,

Polyacrylamidomethyl Benzylidene Camphor manufactured under the name “MEXORYL SW®” by CHIMEX.

Phenyl Benzimidazole Compounds:

Phenylbenzimidazole Sulfonic Acid particularly sold under the trade name “EUSOLEX 232®” by MERCK.

Bis-Benzoazolyl Compounds

Disodium Phenyl Dibenzimidazole Tetrasulfonate sold under the trade name “NEO HELIOPAN AP®” by HAARMANN and REIMER.

Phenyl Benzotriazole Compounds:

Drometrizole Trisiloxane sold under the name “Silatrizole®” by RHODIA CHIMIE.

Methylene Bis-(Hydroxyphenyl Benzotriazole) Compounds

Methylene bis-Benzotriazolyl Tetramethylbutylphenol particularly sold in solid form as the product sold under the trade name “MIXXIM BB/100 C” by FAIRMOUNT CHEMICAL or in the form of an aqueous dispersion of micronized particles with an average particle size varying from 0.01 to 5 μm and preferably from 0.01 to 2 μm and more particularly from 0.020 to 2 μm with at least one alkylpolyglycoside surfactant with a CnH(2n+1) O(C6H10O5)xH structure wherein n is an integer from 8 to 16 and x is the average degree of polymerization of the (C8H10O5) unit and varies from 1.4 to 1.6 as described in patent GB-A-2 303 549 particularly sold under the trade name “TINOSORB M®” by the company BASF or in the form of an aqueous dispersion of micronized particles with an average particle size varying from 0.02 to 2 μm and preferably from 0.01 to 1.5 μm and more particularly from 0.02 to 1 μm in the presence of at least one polyglycerol mono-(C8-C20)alkyl-ester with a degree of polymerization of glycerol of at least such as aqueous dispersions described in application WO2009/063392.

Triazine Compounds:
Bis-Ethylhexyloxyphenol Methoxyphenyl Triazine sold under the trade name “TINOSORB S®” by BASF,
Ethylhexyl Triazone particularly sold under the trade name “UVINUL 25 T150®” by BASF,
Diethylhexyl Butamido Triazone sold under the trade name “UVASORB HEB C” by SIGMA 3V,
2,4,6-tris(dineopentyl 4′-amino benzalmalonate)-s-triazine,
2,4,6-tris(diisobutyl 4′-amino benzalmalonate)-s-triazine,
2,4-bis (n-butyl 4′-aminobenzoate)-6-(aminopropyl trisiloxane)-s-triazine,
2,4-bis(dineopentyl 4′-amino benzalmalonate)-6-(n-butyl 4′-aminobenzoate)-s-triazine,
symmetrical triazine filters substituted by naphthalenyl groups or polyphenyl groups described in U.S. Pat. No. 6,225,467, application WO2004/085412 (see compounds 6 and 9) or document “Symmetrical Triazine Derivatives” IP.COM IPCOM000031257 Journal, INC WEST HENRIETTA, N.Y., US (Sep. 20, 2004) particularly 2,4,6-tris(di-phenyl)-triazine and 2,4,6-tris(ter-phenyl)-triazine that is included in patent applications WO06/035000, WO06/034982, WO06/034991, WO06/035007, WO2006/034992, 40 WO2006/034985, these compounds advantageously being in micronized form (average particle size from 0.02 to 3 μm) that can for example be obtained using the micronization process described in applications GB-A-2 303 549 and EP-A-893119 and particularly in an aqueous dispersion;
triazine silicones substituted by two aminobenzoate groups as described in patent EP0841341 particularly 2,4-bis-(n-butyl 4′-aminobenzalmalonate)-6-[(3-{1, 3, 3, 3-tetramethyl-1-[(trimethylsilyloxy]disiloxanyl}propyl)amino]-s-triazine.

Anthranilic Compounds:

Menthyl anthranilate sold under the trade name “NEO HELIOPAN MA®” by Symrise.

Imidazoline Compounds:

Ethylhexyl Dimethoxybenzylidene Dioxoimidazoline Propionate,

Benzalmalonate Compounds:

Polyorganosiloxane with benzalmalonate functions such as Polysilicone-15 sold under the trade name “PARSOL SLX” by HOFFMANN LA ROCHE.

4,4-Diarylbutadiene Compounds:
1,1-dicarboxy (2,2′-dimethyl-propyl)-4,4-diphenylbutadiene.

Benzoxazole Compounds:

2,4-Bis-[5-1(dimethylpropyl)benzoxazol-2-yl-(4-phenyl)-imino]-6-(2-ethylhexyl)-imino-1, 3, 5-triazine sold under the trade name Uvasorb K2A® by Sigma 3V.

Preferred organic filters are chosen from:

Ethylhexyl Methoxycinnamate

Butylmethoxydibenzoylmethane

Ethylhexyl Salicylate,

Homosalate

Octocrylene

Phenylbenzimidazole Sulfonic Acid,

Benzophenone-3,

Benzophenone-4,

Benzophenone-5,

N-hexyl 2-(4-diethylamino-2-hydroxybenzoyl)-benzoate

4-Methylbenzylidene camphor,

Terephthalylidene Dicamphor Sulfonic Acid,

Disodium Phenyl Dibenzimidazole Tetra-sulfonate,

Methylene bis-Benzotriazolyl Tetramethylbutylphenol

Bis-Ethylhexyloxyphenol Methoxyphenyl Triazine

Ethylhexyl triazone,

Diethylhexyl Butamido Triazone,

2,4,6-tris(dineopentyl 4′-amino benzalmalonate)-s-triazine

2,4,6-tris(diisobutyl 4′-amino benzalmalonate)-s-triazine

2,4-bis-(n-butyl 4′-aminobenzoate)-6-(aminopropyl trisiloxane)-s-triazine

2,4-bis-(dineopentyl 4′-amino benzalmalonate)-6-(n-butyl 4′-aminobenzoate)-s-triazine

2,4-bis-(n-butyl 4′-aminobenzalmalonate)-6-[(3-{1,3,3,3-tetramethyl-1-[(trimethylsilyloxy]disiloxanyl}propyl)amino]-s-triazine,

2,4,6-tris-(di-phenyl)-triazine, 2,4,6-tris-(ter-phenyl)-triazine,

Drometrizole Trisiloxane

Polysilicone-15

1,1-dicarboxy (2,2′-dimethyl-propyl)-4,4-diphenylbutadiene

2,4-bis-[5-1(dimethylpropyl)benzoxazol-2-yl-(4-phenyl)-imino]-6-(2-ethylhexyl)-imino-1,3,5-triazine

and mixtures thereof.

Particularly preferred organic filters are chosen from

Butylmethoxydibenzoylmethane

Ethylhexyl Salicylate,

Homosalate

Octocrylene

N-hexyl 2-(4-diethylamino-2-hydroxybenzoyl)-benzoate

Terephthalylidene Dicamphor Sulfonic Acid,

Bis-Ethylhexyloxyphenol Methoxyphenyl Triazine

Ethylhexyl triazone,

Diethylhexyl Butamido Triazone,

2,4-bis-(n-butyl 4′-aminobenzalmalonate)-6-[(3-{1,3,3,3-tetramethyl-1-[(trimethylsilyloxy]disiloxanyl}propyl)amino]-s-triazine,

Drometrizole Trisiloxane

and mixtures thereof.

The organic filters, when present, are present at contents ranging from 0.01 to 30% by weight and preferably from 0.1 to 20% by weight relative to the total weight of the composition according to the invention.

Compositions according to this invention may also include classical cosmetic additives, chosen particularly from among ionic or non-ionic thickeners, moistening agents, anti-foaming agents, perfumes, preservatives, surfactants, active constituents, fillers, propellants, alkalinizing agents or acidifiers or any other ingredient usually used for cosmetics and/or dermatology.

Thickeners include carboxyvinyl polymers such as Carbopols® (Carbomers) and Pemulens such as Pemulen TR1® and Pemulen TR2® (acrylate/C10-C30 alkylacrylate crosspolymer); polyacrylamides such as for example cross-linked copolymers sold under the names Sepigel 305® (C.T.F.A. name: polyacrylamide/C13-14 isoparaffin/Laureth 7) or Simulgel 600 (C.T.F.A. name: acrylamide/sodium acryloyldimethyltaurate copolymer/isohexadecane/polysorbate 80) by the company Seppic; 2-acrylamido 2-methylpropane sulfonic acid polymers and copolymers, optionally cross-linked and/or neutralized, such as poly(2-acrylamido 2-methylpropane sulfonic acid) marketed by Hoechst under the trade name “Hostacerin AMPS®” (CTFA name: ammonium polyacryloyldimethyl taurate or SIMULGEL 800® marketed by the company SEPPIC (CTFA name: sodium polyacryolyldimethyl taurate/polysorbate 80/sorbitan oleate); 2-acrylamido-2-methylpropane sulfonic acid and hydroxyethyl acrylate copolymers such as SIMULGEL NS® and SEPINOV EMT 10® sold by the company SEPPIC; cellulose derivatives such as hydroxyethylcellulose; polysaccharides and in particular gums such as xanthan gum; water-soluble or water-dispersible silicone derivatives such as acrylic silicones, silicone polyethers and cationic silicones and mixtures thereof.

Surfactants are preferably chosen from among anionic, cationic, non-ionic, zwitterionic and amphoteric surfactants. Preferably, they are chosen from among:
a) non-ionic surfactants, and particularly HLB at or above 8 to 25° C., used alone or in a mixture. Mention can be made in particular of:
   esters and ethers of sugars such as the mixture of cetylstearyl glucoside and cetyl and stearyl alcohols such as Montanov 68 from Seppic;
   oxyethylene and/or oxypropylene ethers (that may comprise from 1 to 150 oxyethylene and/or oxypropylene groups) of glycerol;
   oxyethylene and/or oxypropylene ethers (that may comprise from 1 to 150 oxyethylene and/or oxypropylene groups) of fatty alcohols (particularly C8-C24 and preferably C12-C18 alcohols) such as oxyethylene ether of cetearyl alcohol with 30 oxyethylene groups (CTFA name “Ceteareth-30”), oxyethylene ether of stearyl alcohol with 20 oxyethylene groups (CTFA name “Steareth-20”), oxyethylene ether of the mix of C12-C15 fatty alcohols containing 7 oxyethylene groups (CTFA name “C12-15 Pareth-7”) marketed particularly under the name NEODOL 25-7® by SHELL CHEMICALS;
   esters of polyoxyalkylated fatty acids (particularly polyoxyethylenated and/or polyoxypropylenated) possibly in association with a fatty acid ester and glycerol such as the PEG-100 Stearate/Glyceryl Stearate mixture marketed for example by the company ICI under the trade name Arlacel 165;
   fatty acid esters (particularly C8-C24 acid, and preferably C16-C22 acid) and oxyethylenated and/or oxypropylated glycerol ethers (that may include 1 to 150 oxyethylanated and/or oxypropylenated groups), such as PEG-200 glyceryl monostearate sold particularly under the name Simulsol 220 TM® by the company SEPPIC; PEG-50 stearate and PEG-40 monostearate marketed particularly under the name MYRJ 52P® by the company ICI UNIQUEMA; polyethoxylated glyceryl stearate with 30 ethylene oxide groups such as the TAGAT S® product sold by the company Evonik GOLDSCHMIDT, polyethoxylated glyceryl oleate with 30 ethylene oxide groups like the TAGAT O® product sold by the company Evonik GOLDSCHMIDT, polyethoxylated glyceryl cocoate with 30 ethylene oxide groups like the VARIONIC LI 13® product sold by the company SHEREX, polyethoxylated glyceryl isostearate with 30 ethylene oxide groups such as the TAGAT L® product sold by the company Evonik GOLDSCHMIDT and polyethoxylated glyceryl laurate with 30 groups of ethylene oxide like the TAGAT I® product from the company Evonik GOLDSCHMIDT,
   fatty acid esters (particularly C8-C24 acid and preferably C16-C22 acid) and oxyethylenated and/or oxypropylenated sorbitol ethers (possibly containing 1 to 150 oxyethylenated and/or oxypropylenated groups), such as polysorbate 20 particularly sold under the name Tween 20® by the company CRODA, polysorbate 60 particularly sold under the name Tween 60® by the company CRODA,
   dimethicone copolyol, like that sold under the name Q2-5220® by the company DOW CORNING,
   dimethicone copolyol benzoate (FINSOLV SLB 101® and 201® from the company FINTEX),
      copolymers of propylene oxide and of ethylene oxide, also called OE/OP polycondensates,
   and mixtures thereof.
b) non-ionic surfactants with a HLB less than 8 at 25° C., particularly:
   esters and ethers of sugars such as sucrose stearate, sucrose cocoate, sorbitan stearate and mixtures thereof such as Arlatone 2121® marketed by the company ICI;
   oxyethylene and/or oxypropylene ethers (that may comprise from 1 to 150 oxyethylene and/or oxypropylene groups) of fatty alcohols (particularly C8-C24 and preferably C12-C18 alcohols) such as oxyethylene ether of stearyl alcohol with 2 oxyethylene groups (CTFA name “Steareth-2”),
   esters of fatty acids (particularly C8-C24 acid and preferably C16-C22 acid) and of polyol, in particular of glycerol or of sorbitol, such as glyceryl stearate, such as the product sold under the name TEGIN M® by the company Evonik GOLDSCHMIDT, glyceryl laurate such as the product sold under the name IMWITOR 312® by the company HULS, polyglyceryl-2 stearate, sorbitan tristearate, glyceryl ricinoleate;
   lecithins, such as soy lecithins (such as Emulmetik 100 J from Cargill, or Biophilic H from Lucas Meyer);
   the cyclomethicone/dimethicone copolyol mixture sold under the name Q2-3225C® by the company DOW CORNING.
c) anionic surfactants such as:
   polyoxyethylenated fatty acid salts and particularly those derived from amines or alkaline salts, and mixtures thereof;
   phosphoric esters and their salts such as “DEA oleth-10 phosphate” (Crodafos N 10N from CRODA) or monopotassium monocetyl phosphate or potassium cetyl phosphate (Amphisol K from Givaudan);
   sulfosuccinates such as “Disodium PEG-5 citrate lauryl sulfosuccinate” and “Disodium ricinoleamido MEA sulfosuccinate”;
   alkylethersulfates such as sodium lauryl ether sulfate;
   isethionates;
   acylglutamates such as “Disodium hydrogenated tallow glutamate” (AMISOFT HS-21 R® marketed by AJINOMOTO) and sodium stearoyl glutamate (AMISOFT HS-11 PF® marketed by AJINOMOTO) and mixtures thereof;
   derivatives of soybeans such as potassium soyate;
   citrates, such as Glyceryl stearate citrate (Axol C 62 Pellets from Degussa);
   derivatives of proline, such as Sodium palmitoyl proline (Sepicalm VG from Seppic), or the Mixture of Sodium palmitoyl sarcosinate, Magnesium palmitoyl glutamate, palmitic acid and Palmitoyl proline (Sepifeel One from Seppic);
   lactylates, such as Sodium stearoyl lactylate (Akoline SL from Karlshamns AB);
   sarcosinates, such as sodium palmitoyl sarcosinate (Nikkol sarcosinate PN) or the mixture of Stearoyl sarcosine and Myristoyl sarcosine 75/25 (Crodasin SM from Croda);
   sulfonates, such as Sodium C14-17 alkyl sec sulfonate (Hostapur SAS 60 from Clariant);
   glycinates, such as sodium cocoyl glycinate (Amilite GCS-12 from Ajinomoto).

Among the acidifying agents, mention may be made of mineral or organic acids such as hydrochloric acid, orthophosphoric acid, sulfuric acid, carboxylic acids such as acetic acid, tartaric acid, citric acid, lactic acid, sulfonic acids. Among the alkalinizing agents, mentioned may be made, by way of example, of ammonia, alkaline carbonates, alkanolamines such as mono-, di- and triethanolamines as well as the derivatives thereof, sodium and potassium hydroxides.

Among the active constituents for the treatment of keratin materials such as the skin, lips, scalp, hair, eyelashes or nails, mention may be made for example of:
vitamins and derivatives or precursors thereof, alone or in mixtures,
anti-oxidant agents;
anti-radical agents;
anti-pollutant agents;
self-tanning agents;
anti-glycation agents;
soothing agents,
deodorant agents,
NO-synthase inhibitors;
agents for stimulating the synthesis of dermal or epidermal macromolecules and/or preventing the degradation thereof;
agents for stimulating fibroblast proliferation;
agents for stimulating keratinocyte proliferation;
muscle relaxant agents,
cooling agents,
tightening agents,
matting agents,
moisturizing agents.
anti-inflammatory agents;
antimicrobial agents,
slimming agents,
agents acting on the energy metabolism of cells,
insect repellents,
substance P or CRGP antagonists
anti-hair loss agents
anti-wrinkle agents,
anti-ageing agents,

A person skilled in the art would choose said active constituent(s) according to the effect sought on the skin, hair, eyelashes, eyebrows or nails.

Obviously, a person skilled in the art would take care to select the optional complementary compound(s) cited above and/or the amounts thereof such that the advantageous properties intrinsically associated with the compositions according to the invention are not, or substantially not, impaired by the envisaged addition(s).

The compositions according to the invention may be prepared using the techniques well-known to a person skilled in the art. They may particularly be in the form of a simple or complex emulsion (O/W, W/O, O/W/O or W/O/W) such as a cream, milk or cream gel. They may optionally be packaged in an aerosol and be in the form of foam or spray.

The cosmetic compositions according to the invention may for example be used as a treatment and/or sun protection product for the face and/or body with a liquid to semi-liquid consistency, such as milks, more or less unctuous creams, cream gels, pastes.

The compositions according to the invention in the form of fluid lotions suitable for vaporization according to the invention are applied on the skin or hair in the form of fine particles by means of pressurization devices. The devices suitable for the invention are well-known to those skilled in the art and comprise non-aerosol pumps or “atomizers”, aerosol containers comprising a propellant and aerosol pumps using compressed air as a propellant. The latter are described particularly in U.S. Pat. Nos. 4,077,441 and 4,850,517.

The compositions packaged in an aerosol according to the invention generally contain conventional propellants such as for example the hydrofluorinated compounds dichlorodifluoromethane, difluoroethane, dimethylether, isobutane, n-butane, propane, trichlorofluoromethane. They are preferably present in amounts ranging from 15 to 50% by weight relative to the total weight of the composition.

The present invention further relates to a non-therapeutic cosmetic process for the treatment and/or makeup of a keratin material consisting of applying on the surface of said keratin material at least one composition according to the invention as defined above.

The following examples illustrate the invention without being in any way limitative. In these examples, quantities of the ingredients compositions are given as a % by weight relative to the total weight of the composition.

### EXAMPLES

### Example 1: Preparation of Formulas and Evaluation of the Photo-Instability Thereof

The following formulas 1 to 4 are prepared according to the following protocol:

The aqueous and oily phases are prepared by mixing the raw materials under mechanical stirring at 80° C.; the solutions obtained are macroscopically homogeneous.

The emulsion is prepared by slowly introducing the oily phase into the aqueous phase under stirring using a Moritz type homogenizer at a stirring speed of 4000 RPM for 15 minutes.

The emulsion obtained is cooled to ambient temperature, then the remaining phases are added thereto under slow stirring.

The emulsion is characterized by droplets between 1 μm and 10 μm in size.

The particles of TiO2 of non-inventive example 4 are introduced into the fatty phase, while the particles made from perlite according to the invention and present in formulas 1 to 3 according to the invention are introduced into the emulsion at ambient temperature, therefore after formation of the emulsion.

The photo-instability of formulas 1 to 4 prepared is evaluated as follows:

The photocatalytic effect of TiO2 in the presence of vitamin F in the medium is detected by an increase in pentane formation. In fact, vitamin F is oxidized in UV irradiation to produce pentane.

Mixtures containing vitamin F and particles made from TiO2 were prepared; the control is the mixture with only vitamin F, without TiO2.

The sample is exposed to UV at the dose of 60 J UVA/cm2. The quantity of pentane in the bottle is assayed with and without irradiation. The photo-instability is represented by the following ratio:

Photo-Instability=(% Pentane in the presence of the particle/% pentane without the particle)*100

The particle is considered to be very photo-unstable when the percentage is close to 100%.

The preparation method of the formulas for measuring the photostability of the particles is as follows.

The aqueous phase is heated to 65° C., the emulsion is prepared by slowly introducing the oily phase into the aqueous phase under stirring using a Moritz type homogenizer at a stirring speed of 4000 RPM for 15 minutes. The emulsion obtained is cooled under stirring to 40° C. The emulsion obtained is cooled to ambient temperature, then the remaining phases are added thereto under slow stirring. Vitamin F and the particles are introduced one by one.

The composition of formulas 1 to 4 and the photo-instability results are presented hereinafter:

Compositions 1 to 4 each comprise 2% of TiO2 active material.

| | | Example 1 | Example 2 | Example 3 | Example 4 |
|---|---|---|---|---|---|
| | | According to | According to | According to | Non- |
| | EU INCI | the invention | the invention | the invention | Invention |
| | TRIETHANOLAMINE | 0.55 | 0.55 | 0.55 | 0.55 |
| | CI 77891 (TiO2) | | | | 2 |
| | Particle according to | 10.38 | | | |
| | preparation example A | | | | |
| | coated with 3% silica | | | | |
| | (TITANIUM DIOXIDE | | | | |
| | PERLITE and 3% SILICA) | | | | |
| | Particle according to | | 10.375 | | |
| | preparation example A | | | | |
| | coated with 4% silica | | | | |
| | (TITANIUM DIOXIDE | | | | |
| | PERLITE and 4% SILICA) | | | | |
| | Particle according to | | | 10.38 | |
| | preparation example A | | | | |
| | coated with 5% silica | | | | |
| | (TITANIUM DIOXIDE | | | | |
| | PERLITE and 5% SILICA) | | | | |
| | DIISOPROPYL SEBACATE | 4 | 4 | 4 | 4 |
| | XANTHAN GUM | 0.1 | 0.1 | 0.1 | 0.1 |
| | ACRYLATES/C10-30 ALKYL | 0.25 | 0.25 | 0.25 | 0.25 |
| | ACRYLATE | | | | |
| | CROSSPOLYMER | | | | |
| | (PEMULEN TR-1 POLYMER) | | | | |
| | ALCOHOL DENAT. | 5 | 5 | 5 | 5 |
| | Water | Qsp100 | Qsp100 | Qsp100 | Qsp100 |
| | C12-15 ALKYL BENZOATE | 13 | 13 | 13 | 13 |
| | GLYCERIN | 7 | 7 | 7 | 7 |
| | STEARIC ACID | 1 | 1 | 1 | 1 |
| | GLYCERYL STEARATE | 2 | 2 | 2 | 2 |
| | (and) PEG-100 STEARATE | | | | |
| | (ARLACEL 165-FP-PA-(SG)) | | | | |
| | POTASSIUM CETYL | 1 | 1 | 1 | 1 |
| | PHOSPHATE | | | | |
| | (AMPHISOL K) | | | | |
| | LINOLEIC ACID (and) OLEIC | 6 | 6 | 6 | 6 |
| | ACID (and) LINOLENIC ACID | | | | |
| | (VITAMIN F ACID) | | | | |
| Photo-Instability | | 72% | 35% | 14% | 100% |
| 60J | | | | | |

The results obtained therefore demonstrate that the TiO2 contained in a perlite matrix and coated with silica according to the invention is more photostable, and that this photostability increases according to the silica coating rate.

### Example 2: Preparation of Formulas and Evaluation of the Stability Thereof and of Whitening on Application

The following formulas 5 to 8 were prepared as described in example 1.

### The Stability of Formulas 5 to 8 is Evaluated as Follows:

The stability of the compositions according to the invention is evaluated by means of microscopic observations of the appearance thereof, specifically the state of dispersion of the particles. A composition is deemed stable when the microscopic appearance thereof and the viscosity thereof are stable at T0 and for 1 month at ambient temperature (25° C.).

### The Whitening of Formulas 5 to 8 is Evaluated as Follows:

The whitening of the formulas is evaluated according to the following method:

2 mg of the formula is deposited on a black fabric surface with a similar roughness to skin. The product is spread by applying a circular movement of 10 revolutions. The product is dried for 5 min and the whitening is evaluated, with a score of +++ for the most whitening and + for the least whitening.

The composition of formulas 5 to 8 and the instability and whitening results are presented hereinafter:

| | Example 5 | Example 6 | Example 7 | Example 8 |
|---|---|---|---|---|
| | Non- | According to | According to | According to |
| EU INCI | Invention | the invention | the invention | the invention |
| DISODIUM EDTA | 0.1 | 0.2 | 0.2 | 0.2 |
| TOCOPHEROL | 0.1 | 0.1 | 0.1 | 0.1 |
| TRIETHANOLAMINE | 0.71 | 0.71 | 0.71 | 0.71 |
| Preservatives | Qs | Qs | Qs | Qs |
| ISOHEXADECANE | 4.5 | 4.5 | 4.5 | 4.5 |
| BUTYL | 3 | 3 | 3 | 3 |
| METHOXYDIBENZOYLMETHANE | | | | |
| (PARSOL 1789) | | | | |
| TITANIUM DIOXIDE (and) | 3 | | | |
| ALUMINUM HYDROXIDE | | | | |
| (and) STEARIC ACID | | | | |
| (MICRO TITANIUM | | | | |
| DIOXIDE MT-100 T V) | | | | |
| ETHYLHEXYL TRIAZONE | 0.5 | 0.5 | 0.5 | 0.5 |
| (UVINUL T 150) | | | | |
| TEREPHTHALYLIDENE | 0.9 | 0.9 | 0.9 | 0.9 |
| DICAMPHOR SULFONIC | | | | |
| ACID | | | | |
| (MEXORYL SX) | | | | |
| OCTOCRYLENE | 7 | 7 | 7 | 7 |
| (SUNOBEL OCT) | | | | |
| Particle according to | | 6.23 | | |
| preparation example A | | | | |
| coated with 3% silica | | | | |
| (TITANIUM DIOXIDE | | | | |
| PERLITE and 3% SILICA) | | | | |
| Particle according to | | | 6.23 | |
| preparation example A | | | | |
| coated with 4% silica | | | | |
| (TITANIUM DIOXIDE | | | | |
| PERLITE and 4% SILICA) | | | | |
| Particle according to | | | | 6.23 |
| preparation example A | | | | |
| coated with 5% silica | | | | |
| (TITANIUM DIOXIDE | | | | |
| PERLITE and 5% SILICA) | | | | |
| XANTHAN GUM | 0.1 | 0.1 | 0.1 | 0.1 |
| ACRYLATES/C10-30 | 0.25 | 0.25 | 0.25 | 0.25 |
| ALKYL ACRYLATE | | | | |
| CROSSPOLYMER | | | | |
| (PEMULEN TR- | | | | |
| 1 POLYMER) | | | | |
| DIMETHICONE | 0.5 | 0.5 | 0.5 | 0.5 |
| (BELSIL DM 350) | | | | |
| ALCOHOL DENAT. | 2 | 2 | 2 | 2 |
| Water | Qsp100 | Qsp100 | Qsp100 | Qsp100 |
| C12-15 ALKYL BENZOATE | 7.5 | 7.5 | 7.5 | 7.5 |
| (FINSOLV TN) | | | | |
| GLYCERIN | 6 | 6 | 6 | 6 |
| PROPYLENE GLYCOL | 6 | 6 | 6 | 6 |
| STEARIC ACID | 1 | 1 | 1 | 1 |
| GLYCERYL STEARATE | 1 | 1 | 1 | 1 |
| (and) PEG-100 STEARATE | | | | |
| (SIMULSOL 165) | | | | |
| POTASSIUM CETYL | 1 | 1 | 1 | 1 |
| PHOSPHATE | | | | |
| (AMPHISOL K) | | | | |
| Whitening | +++ | + | + | + |
| Stability 2 months 40° C. | Stable | Stable | stable | stable |
| and 45° C. | | | | |

The results show the in vitro whitening measurements of formulas comprising 2.5% by weight of TiO2 active material.

On the other hand, the whitening is lowest with the particles according to the invention (formulas 6 to 8).

Furthermore, the compositions according to the invention have a high SPF and high persistent pigmentation darkening (or PPD, targeted on UVA).

1. A composition comprising:
a) at least one aqueous phase; and
b) particles with a number-average elementary size greater than 0.1 μm comprising:
i) a perlite matrix,
ii) at least one inorganic UV filter, and
iii) a silica coating, said silica representing at least 1% by weight relative to the total weight of the composite particle.

2. The composition according to claim 1, wherein the composite particles have a number-average elementary size varying between 0.1 and 30 μm.

3. The composition according to claim 1, wherein the composite particles are present in concentrations ranging from 1 to 70 by weight relative to the total weight of the composition.

4. The composition according to claim 1, wherein the inorganic UV filter is chosen among metal oxides, preferably titanium, zinc, iron oxides or mixtures thereof.

5. The composition according to claim 1, wherein the inorganic UV filter is present in the composite particles in a content ranging from 1% to 70% by weight relative to the total weight of a composite particle.

6. The composition according to claim 1, wherein perlite is present in the composite particles in a content ranging from 10 to 99% by weight relative to the total weight of a composite particle.

7. The composition according to claim 1, wherein the silica coating represents at least 2% by weight relative to the total weight of a composite particle.

8. The composition according to claim 1, wherein the silica coating represents from 1 to 10% by weight relative to the total weight of a composite particle.

9. The composition according to claim 1, wherein the aqueous phase represents from 15 to 95% by weight relative to the total weight of the composition.

10. The composition according to claim 1, wherein comprises at least one oily phase.

11. The composition according to claim 1, which comprises at least one organic UV filter.

12. A cosmetic process for the treatment and/or makeup of human keratin materials, comprising at least the application on said keratin materials of a composition according to claim 1.

13. The composition according to claim 2, wherein the composite particles are present in concentrations ranging from 1 to 70% weight relative to the total weight of the composition.

14. The composition according to claim 2, wherein the inorganic UV filter is chosen among metal oxides, preferably titanium, zinc, iron oxides or mixtures thereof.

15. The composition according to claim 3, wherein the inorganic UV filter is chosen among metal oxides, preferably titanium, zinc, iron oxides or mixtures thereof

16. The composition according to claim 2, the inorganic UV filter is present in the composite particles in a content ranging from 1% to 70% by weight relative to the total weight of a composite particle.

17. The composition according to claim 3, the inorganic UV filter is present in the composite particles in a content ranging from 1% to 70% by weight relative to the total weight of a composite particle.

18. The composition according to claim 4, the inorganic UV filter is present in the composite particles in a content ranging from 1% to 70% by weight relative to the total weight of a composite particle.

19. The composition according to claim 2, wherein perlite is present in the composite particles in a content ranging from 10 to 99% by weight by weight relative to the total weight of a composite particle.

20. The composition according to claim 3, wherein perlite is present in the composite particles in a content ranging from 10 to 99% by weight by weight relative to the total weight of a composite particle.
